(19) United States
(12) Patent Application Publication
(10) Pub. No.: US 2026/0335521 A1
(43) Pub. Date: Oct. 29, 2026

(54) SYSTEMS AND METHODS FOR OXYGENATING BLOOD, PASSIVE OXYGENATION CIRCUITS, AND NEONATAL EXTRACORPOREAL SUPPORT SYSTEMS

(71) Applicants: THE CHILDREN'S HOSPITAL OF PHILADELPHIA, Philadelphia, PA (US); VITARA BIOMEDICAL, INC., Philadelphia, PA (US)

(72) Inventors: Marcus Graeme DAVEY, Ardmore, PA (US); Christopher C. Gregory, Newtown, PA (US); James S. MCGLONE, Newtown, PA (US); Alan W. FLAKE, Philadelphia, PA (US)

(21) Appl. No.: 19/111,634

(22) PCT Filed: Sep. 14, 2022
(86) PCT No.: PCT/US2022/043509

§ 371 Date: Mar. 13, 2025

(51) Int. Cl. A61M 1/16; A61M 1/36

(52) U.S. Cl. CPC A61M 1/1698; A61M 1/367; A61M 2202/0208; A61M 2202/0225; A61M 2202/0462; A61M 2205/3334; A61M 2230/04; A61M 2240/00

(57) ABSTRACT

A method of oxygenating neonatal blood can include receiving deoxygenated arterial blood from an umbilical cord of a neonate within a pumpless extracorporeal circuit. The extracorporeal circuit can include a membrane oxygenator. The deoxygenated arterial blood can have a blood-flow rate RF. The method can include flowing a blended sweep gas through the membrane oxygenator at a blended sweep gas flow rate of at least 2 R_{F} or greater. The blended sweep gas can comprise oxygen and at least about 1% carbon dioxide by volume.

### BACKGROUND OF THE INVENTION

Extracorporeal support has been proposed both for adults and neonates. Such systems utilize an oxygenator to exchange oxygen and carbon dioxide with blood from the patient's body.

### SUMMARY

A method of oxygenating neonatal blood can include receiving deoxygenated arterial blood from an umbilical cord of a neonate within a pumpless extracorporeal circuit. The extracorporeal circuit can include a membrane oxygenator. The deoxygenated arterial blood can have a blood-flow rate RF. The method can include flowing a blended sweep gas through the membrane oxygenator at a blended sweep gas flow rate of at least 2 R_{F} or greater. The blended sweep gas can comprise oxygen and at least about 1% carbon dioxide by volume.

The blended sweep gas can include one or more selected from the group consisting of: ambient air at sea level, nitrogen, and nitric oxide. The blended sweep gas can include a sufficient partial pressure of carbon dioxide to maintain normocapnia in the neonate's venous blood. The blended sweep gas flow rate can be between about 2 R_{F} and about 20 R_{F}.

The blended sweep gas flow rate can be between about 2 R_{F} and about 16 R_{F}. The method can include measuring the blood-flow rate R_{F}. The blended sweep gas flow rate can be between about 120 mL/minute and about 800 mL/minute. The blended sweep gas can include between about 3% and about 6% carbon dioxide by volume. At least a portion of the carbon dioxide of the blended sweep gas can be previously recovered from exhaust gas from the membrane oxygenator.

The method can include receiving with a gas blender, a first gas from a first source and a second gas from a second source, the first gas different from the second gas, and blending the first gas and the second gas to form the blended sweep gas. The first source can be a first gas tank and the second source can be a second gas tank. The first source can be a wall line and the second source can be at least one of a second wall line and a gas tank.

A pumpless oxygenation circuit can include a mixer adapted configured to receive oxygen-containing gas from an oxygen source, receive carbon-dioxide-containing gas from a carbon-dioxide source, output a blended sweep gas comprising oxygen and at least about 1% carbon dioxide by volume. The pumpless oxygenation circuit can include a flow controller, a membrane oxygenator, and a blood-flow sensor. The flow controller can be fluidically coupled to the output of the mixer, the flow controller adapted to permit a blended sweep gas flow rate of the blended sweep gas through the flow controller. The membrane oxygenator can be adapted for connecting to the flow controller and a pumpless neonatal blood circuit. The blood-flow sensor can be positioned along the pumpless neonatal blood circuit, the blood-flow sensor adapted and configured to measure a neonatal-blood-flow rate R_{F}. The flow controller can control the blended sweep gas flow rate to be at least 2 R_{F} or greater.

The pumpless oxygenation circuit can include a controller communicatively coupled with the flow controller and the blood-flow sensor. The pumpless oxygenation circuit can include a blood-gas sensor positioned along the pumpless neonatal blood circuit, the blood-gas sensor communicatively coupled to the controller. The controller can control the mixer to titrate a composition of the blended sweep gas to reflect neonatal blood-gas values and provide a sufficient partial pressure of carbon dioxide to maintain normocapnia. The blended sweep gas flow rate can be between about 2 R_{F} and about 20 R_{F}. The blended sweep gas flow rate can be between about 2 R_{F} and about 16 R_{F}. The blended sweep gas flow rate can be between about 120 mL/minute and about 800 mL/minute. The blended sweep gas can include between about 3% and about 6% carbon dioxide by volume.

A neonatal extracorporeal support system can include a neonatal chamber, a neonatal blood system, and a pumpless oxygenation circuit. The neonatal chamber can be adapted and configured hold a neonate in a physiological saline solution. The neonatal blood system can be adapted and configured for coupling to a plurality of blood vessels of a neonate.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a schematic of an extracorporeal support system according to an aspect of the disclosure.

FIG. 2 illustrates a schematic of a portion of an extracorporeal support system according to another aspect of the disclosure.

FIG. 3 illustrates an isometric view of a portion of an extracorporeal support system according to yet another aspect of the disclosure.

FIG. 4 illustrates an isometric view of an oxygenator according to an aspect of the disclosure.

FIG. 5 illustrates an isometric view of a portion of a gas exchanger according to an aspect of the disclosure.

FIG. 6 illustrates an isometric view of a gas exchanger according to another aspect of the disclosure.

FIG. 7 illustrates a front elevation view of the gas exchanger of FIG. 6.

FIG. 8 illustrates a side elevation view of an oxygenator according to an aspect of the disclosure.

FIG. 9 illustrates a method of oxygenating blood according to an aspect of the disclosure.

FIG. 10 illustrates a system for oxygenating blood according to an aspect of the disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing.

The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Aspects of the invention provide methods and systems for oxygenating blood.

Aspects of the invention can utilize a high flow of CO₂-rich sweep gas. Such aspects avoid the technical challenges of regulating small flow rates of oxygen-rich sweep gas posed by the low neonatal blood flow rate.

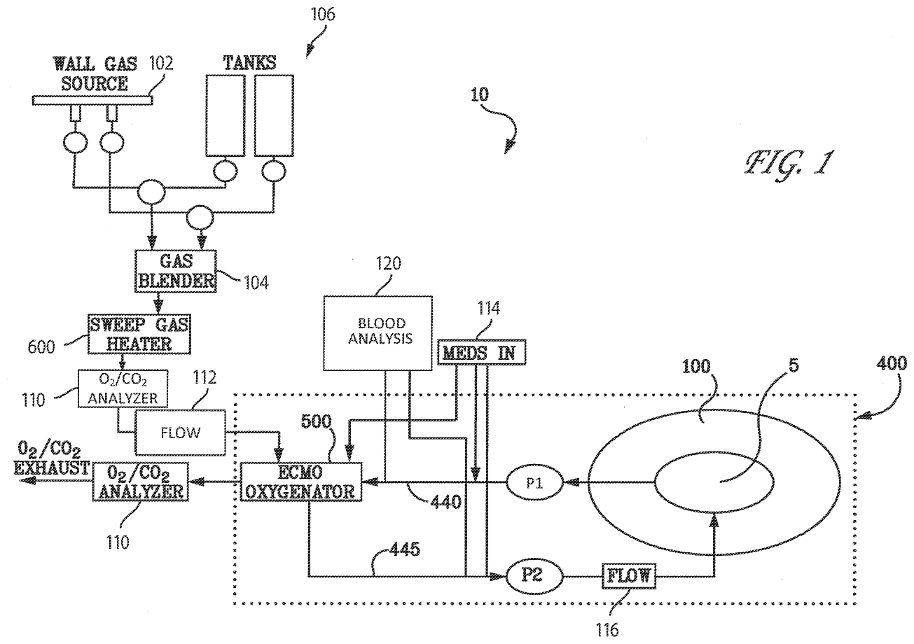

Referring now to FIG. 1, one aspect of the invention provides an extracorporeal support system particularly useful for neonates.

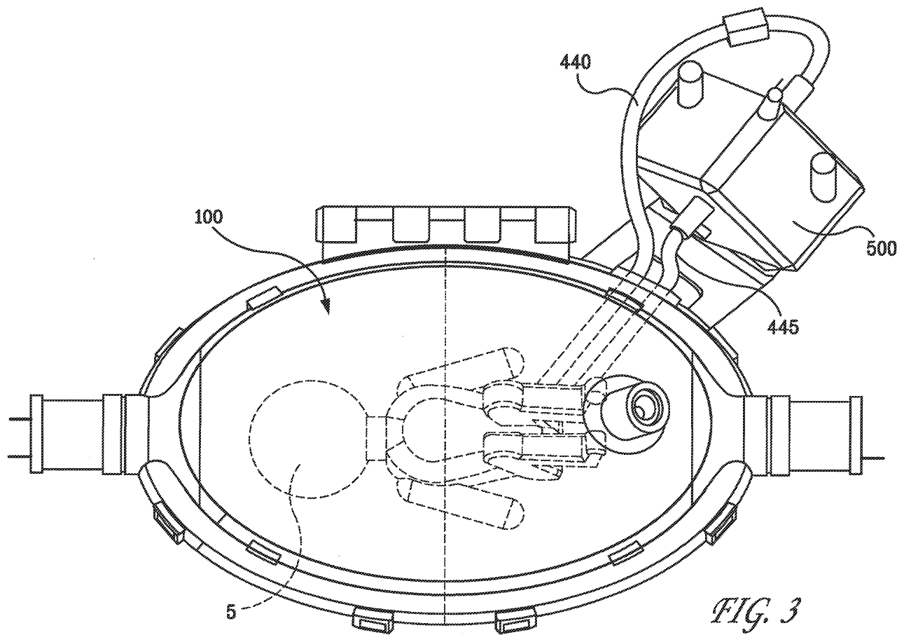

Referring to FIGS. 1-3, a system 10 is configured to provide extracorporeal support to a neonate. According to one aspect of the disclosure, the system 10 may be configured to provide a system environment that is similar to an environment the neonate would experience in utero. Viability of a neonate that is removed from the uterine environment (e.g., due to preterm birth) and that is, for example, between about 22 weeks to about 28 weeks gestation, may be increased by placing the neonate in the system environment.

According to an aspect of the disclosure, the system environment may be configured to accomplish one or more of the following objectives: (1) limit exposure of the neonate to light; (2) limit exposure of the neonate to sound; (3) maintain the neonate submerged within a liquid environment; (4) maintain the neonate within a desired temperature range; (5) expand to accommodate the growth of the neonate; and/or (6) control the oxygenation level within the neonate's blood stream. The system also permits neonatal activities (e.g., neonatal breathing movements, neonatal swallowing of fluid) necessary for organ growth and development.

The system 10 may be configured to treat neonates (e.g., less than 37 weeks estimated gestational age, particularly about 28 to about 32 weeks estimated gestational age), or extreme premature neonates (about 22 to about 28 weeks estimated gestational age). The gestation periods are provided for humans, though corresponding preterm neonates of other animals may be used. In a particular embodiment, the neonate has no underlying congenital disease. The term or preterm neonate may have limited capacity for pulmonary gas exchange, for example, due to pulmonary hypoplasia or a congenital anomaly affecting lung development, such as congenital diaphragmatic hernia. In a particular aspect, the subject may be a preterm or term neonate awaiting lung transplantation, for example, due to congenital pulmonary disease (e.g., bronchoalveolar dysplasia, surfactant protein B deficiency, and the like). Such transplantation surgeries are currently rarely performed in the United States. However, the number of transplantation surgeries may be increased with the more stable method for pulmonary support provided by the instant invention. The neonate 5 may also be a candidate for ex utero intrapartum treatment (EXIT) delivery, including patients with severe airway lesions and a long-expected course before definitive resection. The neonate 5 may also be a neonatal surgical or fetoscopic procedure patient, particularly with preterm labor precipitating early delivery.

According to one aspect of the disclosure, the system 10 may be configured such that the neonate 5 is maintained in the system 10 for a selected time period (for example, for days, weeks or months, until the neonate 5 is capable of life without the system 10). The system 10 should be operable to maintain the neonate 5 for at least 7 days, at least 14 days, at least 21 days, at least 28 days, at least 35 days, at least 42 days, at least 49 days, or at least 56 days.

The system 10 includes a neonatal chamber 100 configured to house a neonate 5, a physiologic saline solution (PSS) circuit configured to provide a flow (e.g., a constant flow) of PSS through the neonatal chamber 100, and an oxygenation circuit 400 configured to remove carbon dioxide from the neonate's blood and supply oxygen to the neonate's blood.

The system 10 is configured to maintain the neonate 5 in the neonatal chamber 100 immersed in PSS. The system 10 is further configured such that the oxygenation circuit 400 provides adequate gas exchange for the neonate 5 to sustain life. In this way, the system 10 provides an environment similar to an intrauterine environment to facilitate continued growth and development of the neonate 5. The system 10 may include a cart or similar device (not shown) that facilitates monitoring, caring for, and transporting the neonate 5 within a medical facility.

According to an aspect of this disclosure, the system 10 may be as described in pending U.S. Patent Application Publication No. 2019/0380900, which is USSN Ser. No. 16/469,192.

The oxygenation circuit 400 can be connected with the neonate 5 in a venous/venous arrangement. Alternatively, the oxygenation circuit 400 may be connected with the neonate 5 in an arterial/venous arrangement. Cannulas may be placed in the great neck vessels (e.g., carotid, jugular) of the neonate 5 to connect the circulatory system of the neonate 5 to the oxygenator 500. The placement in the great neck vessels may avoid issues of vasospasm and cannula instability in umbilical vessels. An external portion of the cannulas may be fitted with a sleeve (e.g., to permit increased tension of the stabilizing sutures). The sleeve may be made of silicone and may range, for example, from about 1 to about 10 cm in length, or particularly from about 3 to about 5 cm in length. The cannulas may be sutured to the neonate 5 (for example via the fitted sleeve) to secure the cannulas to the neck of the neonate 5.

In some aspects, the oxygenation circuit 400 may be connected to the neonate 5 via the neonate's umbilical cord. In such an arrangement, cannulas may be sutured or connected via methods and/or devices into the veins and arteries of the umbilical cord. It will be appreciated that other connection arrangements may be utilized. In one particular embodiment, a non-suturing device or cannula is described in US Publ. No. 2021/0338270. In this or other embodiments, the neonate 5 is connected to the oxygenation circuit 400 using a cannula which connects the one vein and two arteries of the neonate 5 umbilical cord.

The oxygenation circuit 400 may include an oxygenator 500 (shown in FIG. 1 as an extracorporeal membrane oxygenation (ECMO)) for providing gas exchange functionality, particularly of oxygen (to) and carbon dioxide (from), to the neonate 5. One oxygenator contemplated for use is described in PCT application number filed Sep. 14, 2022 titled “Oxygenating and Neonatal Extracorporeal Support Devices and Systems” the disclosure of which is hereby incorporated by reference as if set forth in its entirety herein. The oxygenator 500 can be removably connected to the neonate 5 and, optionally, to other components of the oxygenation circuit 400 and the system 10. The oxygenator 500 is connected with the neonate 5 via two or more fluid lines and includes at least a drain line 440 and an inlet line 445. Blood flows from the neonate 5 though the drain line 440 to the oxygenator 500. The system 10 can include a medication supply line 114 that introduces medication into the neonate blood in the drain line 440. A sensor 120 can analyze blood flow through the drain line. The sensor 120 can also perform a gas analysis on the sweep gas returning to the oxygenator 500 in the drain line 440. The medication supply line 114 can also introduce medication into the neonate blood in the inlet line 445. Medication can also be introduced into the oxygenator 500 though the medication supply line 114.

The blood then flows through the oxygenator 500 and returns to the neonate 5 via the inlet line 445. The oxygenation level of the blood that flows out of the oxygenator and in through the inlet line 445 are substantially equivalent. In certain embodiments, the oxygenation level of the blood is measured using a gas analyzer 110, such as a ML206 Gas Analyzer from AD Instruments, which uses an infrared sensor and an optical or visible spectrum absorption to measure the levels of CO₂ and O₂ in the blood, respectively. A first gas analyzer 110 can analyze sweep gas flowing to the oxygenator 500. A second gas analyzer 110 can analyze oxygenation levels in the blood. A sampler 112 can sample flow rate of the sweep gas. In this or other embodiments, the sampling pump flow rate ranges from about 35 to about 200 milliliter per minute (ml/min). The remaining gas within the blood comprises nitrogen (N₂).

In some aspects, the oxygenator 500 may be configured to be disconnected and replaced with another oxygenator 500 while the oxygenation circuit 400 is operational. If the oxygenator 500 is damaged or has surpassed its expected life cycle (typically 8 hours or such other time period(s) based on regulatory approvals), the oxygenation circuit 400 may be temporarily configurable to bypass the oxygenator 500 so that the oxygenator 500 may be disconnected from the oxygenation circuit 400 and a new, primed, oxygenator 500 connected in its place without interruption of blood flow.

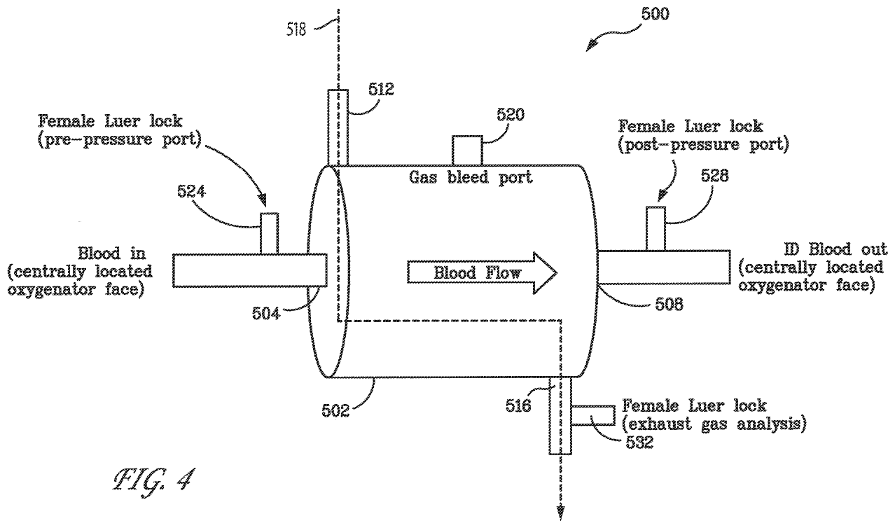

Referring to FIGS. 4-8, the oxygenator 500 includes a housing 502 that defines a cavity 540 therein. The housing 502 may include a plurality of ports that extend through the housing 502 into the cavity 540. A blood inlet port 504, at which blood from the neonate 5 can enter the oxygenator 500, is disposed on the housing 502 and is in fluid communication with cavity 540 and a blood output port 508. Housing 502 has an interior volume to house the gas exchanger 550. In some aspects, multiple blood inlet ports 504 may be configured to receive, either alternatingly or simultaneously, blood from the neonate 5. The blood inlet port 504 is connected to drain line 440, through which the blood moves from the neonate 5 to the oxygenator 500.

One or more additional ports, such as a pressure transducer 524, may be disposed on or adjacent to the blood inlet port 504 or in-line with the drain line 440. The pressure transducer 524 can measure the pressure of the blood from the neonate 5 that enters the oxygenator 500 at the blood inlet port 504. In some aspects, a sampling port (not shown) may also be disposed on or adjacent to the blood inlet port 504 or the drain line 440 to allow for a portion of the blood entering the oxygenator 500 to be removed from the oxygenation circuit 400 to be analyzed or tested. The sampling port may also be used to inject or infuse medicine or nutrition, such as total perinatal nutrition (TPN) directly into the blood. The one or more additional ports may have any suitable connection means, such as a Luer connector.

A blood outlet port 508, through which the blood leaves the oxygenator 500 and is returned to the neonate 5, is disposed on the housing 502. The blood outlet port 508 is connected to the inlet line 445, through which the blood moves from the oxygenator 500 to the neonate 5. The number of blood outlet ports 508 may be equal to the number of blood inlet ports 504, or it may be different.

One or more additional ports, such as a pressure transducer 528, may be disposed on or adjacent to the blood outlet port 508 or in-line with the inlet line 445. The pressure transducer 528 can measure the pressure of the blood exiting the oxygenator 500. In some aspects, a sampling port (not shown) may also be disposed on or adjacent to the blood outlet port 508 or the inlet line 445 to allow for a portion of the blood exiting the oxygenator 500 to be removed from the oxygenation circuit 400 to be analyzed or tested. The sampling port at the exit of the oxygenator 500 (e.g., located on the line from the outlet port 508 or on the line with the pressure transducer 528 (port located before the transducer 528 preferably) may also be used to inject or infuse medicine or nutrition directly into the blood. The one or more additional ports may have any suitable connection means, such as a Luer connector.

A fluid flow meter 116 (FIG. 1) may be positioned in-line with the inlet line 445 to monitor the flow rate of the blood returning to the neonate 5 or exiting from the oxygenator 500.

A gas inlet port 512 is disposed on the housing 502 for introducing a sweep gas into the oxygenator 500. The sweep gas can flow along a direction 518 from the gas inlet port 512 to a gas outlet 516. The sweep gas may comprise a single gas or a combination of various gases, for example oxygen and other environmental gases. It will be appreciated that the sweep gas may comprise various ratios of gases that may be adjusted to achieve a desired combination and ratio of gases for use with system 10. In some aspects, the sweep gas may have a flow rate ranging from about 25 mL/min to about 300 mL/min, from about 25 mL/min to about 200 mL/min, from about 50 mL/min to about 175 mL/min, or from about 75 mL/min to about 150 mL/min. In some aspects, the sweep gas flow rate is about 100 mL/min. An additional port (not shown) may be disposed on or adjacent to the gas inlet port 512, and a portion of the sweep gas entering the oxygenator 500 may be removed for analysis or testing. The additional port may have any suitable connection means, such as a Luer connector. In one embodiment, the flow rate is adjusted from entry at 524 to exit at 528 by partially closing the caliber of the tubing (or reducing the diameter of the tubing) to increase the resistance to slow the flow therethrough. In this or other embodiments, the resistance of oxygenator inherently controls the gas flow. The neonate can regulate the gas flow by increasing heart rate. The flow rate can be sensed via a sensor. In some embodiments, the sensor is a pressure sensor. In other embodiments, the sensor is a velocity flow sensor, volumetric flow meter, or mass flow meter.

A gas exhaust port 516 is disposed on the housing 502 for emitting the sweep gas from the oxygenator 500. An additional port (not shown) may be disposed on or adjacent to the gas exhaust port 516, and a portion of the sweep gas exiting the oxygenator 500 may be removed for analysis or testing. The additional port may have any suitable connection means, such as a Luer connector.

A gas bleed port 520 may be disposed on the housing 502 for removing excess gas when the oxygenator is filled with fluid. If the pressure is too great inside the oxygenator 500, the flow of blood into the oxygenator 500 may be obstructed, slowed, or stagnated, which can result in unwanted clotting and/or poor blood circulation for the neonate 5. Unwanted pressure build-up inside the oxygenator 500 may also increase pressure acting on the blood exiting the oxygenator 500 and flowing to the neonate 5. This may increase the flow rate of the blood, which can result in damage to the blood (e.g., to the hemocytes in the blood), leading to unwanted clot formation and decreased blood quality.

In some aspects, the oxygenation circuit 400 is configured such that the blood moves therethrough without actuation from an external pump (e.g., a mechanical pump). Instead, blood is circulated through the drain line 440, the oxygenator 500, the inlet line 445, and any other components by the neonate's heart. That is, the oxygenation circuit 400 is a passive or a pumpless circuit.

As such, it is advantageous to minimize pressures and resistance within the oxygenation circuit 400, and particularly within the oxygenator 500, so that the blood can be moved therethrough without excess obstruction. The use of a pumpless system avoids exposure of the neonate's heart to excess preload encountered in non-pulsatile pump-assisted circuits. The pumpless system also permits intrinsic neonatal circulatory regulation of flow dynamics. The oxygenator 500 preferably has at least one or more of the following characteristics: very low resistance, low priming volume, low transmembrane pressure drops, and provides efficient gas exchange. Unwanted pressure build-up in the oxygenator 500, as described above, can also require additional force for moving the blood therethrough. This may put strain on the neonate's heart, leading to health complications. If the heart is unable to overcome the added forces, blood flow may stagnate or slow down significantly, which would lead to stopped or decreased circulation of blood in the neonate.

In some aspects, the oxygenator 500 may have a fluid pressure drop measured across the oxygenator inlet 504 and outlet 508 of less than about 50 mmHg, less than about 40 mmHg, or less than about 30 mmHg at 1.5 L/min of blood flow. To achieve a flow rate ranging from about. 50 ml to 200 ml flow in for a neonate, the fluid pressure drop is about 10 mmHg or less. so pressure drop is less than 10 mmHg. Key-low resistance oxygenator. In some aspects, the neonatal blood pressure may be between ranges from about 20 mmHg and to about 40 mmHg. The priming volume of the oxygenator 500 may be less range from about 20 mL to about 200 mL, or from about 30 mL to about 100 mL, or from about 40 mL to about 85 mL or from about 50 mL to about 75 mL. than about 200 100 mL, less than about 85 mL, less than about 75 mL, less than about 50 mL, less than about 40 mL, or less than about 30 mL. In some aspects, it may be preferable to have a priming volume between about ranging from about 20 mL and to about 50 mL or between from about 20 mL and to about 40 mL or from about 25 mL to about 30 mL. Such a small priming volume is advantageous because it decreases dilution of the neonate's blood with that of the priming material. In some aspects, the oxygenator 500 may have a blood flow rate [of ranging from about at least about 1.5 L/min, and preferably between to about 1.52.8 L/min and from about 2.0 L/min, or about 1.5 L/min to about 2.5 0 L/min, or 1.5 L/min to about 2.8 L/min or greater. In certain embodiments, the oxygenator 500 may have a gas transfer rate ranging from about 30 to about 180 mL/min, about 25 to about 50 mL/min, about 50 to about 75 mL/min, about 75 to about 100 mL/min, about 100 to about 150mL/min, about 150 mL/min to about 180 mL/min or greater for a sweep gas comprising oxygen gas (O2). However, the gas transfer rate is not limited to such ranges. In these or other embodiments, the gas transfer rate can be the rate at which gas is transferred into the blood of the neonate. In some embodiments, the composition of the gas can influence the gas transfer rate. The blood flow rate through the oxygenator can influence the gas transfer rate. In some embodiments, a higher blood flow rate can increase the gas transfer rate.

The oxygenator 500 includes a gas exchanger 550 disposed within the cavity 540. The blood that enters the cavity 540 at the blood inlet port 504 contacts and flows through and past the gas exchanger 550. The blood then exits the cavity 540 via the blood outlet port 508 on the housing 502. The gas exchanger 550 includes an element configured to allow at least oxygen and carbon dioxide gases to diffuse between the gas exchanger 550 and the blood flowing through the oxygenator 500. The gas exchanger 550 may include a plurality of hollow fibers 554 arranged in a pattern, such that the blood may flow past the fibers 554 while contacting at least a portion of and/or passing near to but not contacting the fibers 554. As the blood contacts and/or passes near to the fibers 554, diffusion of gases is permitted to occur. It will be appreciated that the rate of diffusion may be predetermined and controlled by at least one or more of the following variables: the composition of the sweep gas, the rate of blood flow, the rate sweep gas flow, the quantity of the fibers 554, the size and shape of the fibers 554, the relative spacing of the fibers 554 within the oxygenator 500 or chamber 502, the blood flow path within the oxygenator, the sweep gas flow path within the oxygenator, and/or by other factors that can affect the above variables.

As shown in FIG. 1, two or more gases, can be blended together in a gas blender 104 to form the sweep gas. In one embodiment, the gases are comprised of oxygen and ambient air. The gas blender can take a variety of forms including a simple wye fitting. A mass flow controller upstream from the gas blender can control the ratio of the gasses contained within the sweep gas. The two or more gases may be supplied by a first supply 102. The first supply 102 can be a high-volume gas reservoir, such as wall lines connected with a central gas supply configured to provide gas to the reservoir. Such gas configurations may be dependent upon the utility supply and configuration on the health care facilities. Alternatively, the two or more gases may be supplied from a second supply 106. The second supply 106 can be gas reservoirs. In one embodiment, the second supply 106 includes a portable oxygen tank and a portable air tank or other gas vessel. It will be appreciated that a variety of suitable gases may be used. In some aspects, oxygen and nitrogen gases may be blended to achieve the desired concentration of oxygen. The oxygen concentration may range from 0% to 100% of the blended gas combination.

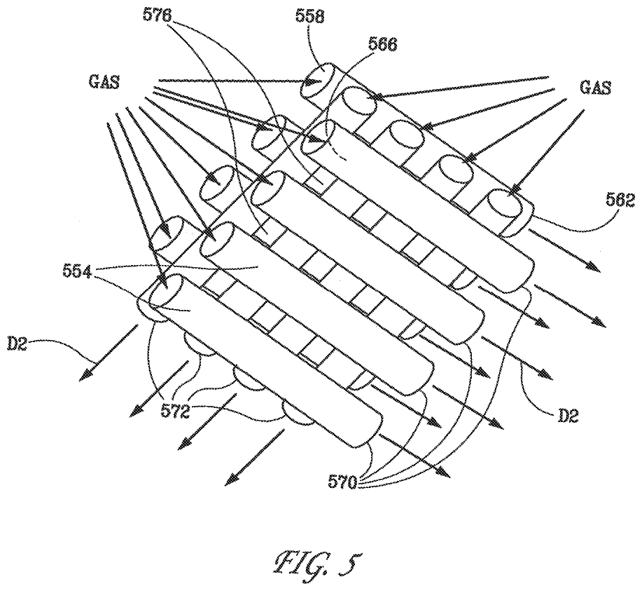

In some aspects such as the embodiment shown in FIG. 5, the fibers 554 comprise polymethylpentene (PMP) due to PMP's desirable qualities of gas permeability. Other suitable gas-permeable materials such as, but not limited to, silicon sheet membranes or polypropylene hollow fiber may be used. Each fiber 554 may have a receiving end 558, at which the sweep gas can enter the fiber 554, and an emitting end 562, from which the sweep gas exits the fiber 554. A channel 566 extends between the receiving end 558 and the emitting end 562 and is configured to carry the sweep gas through the fiber 554.

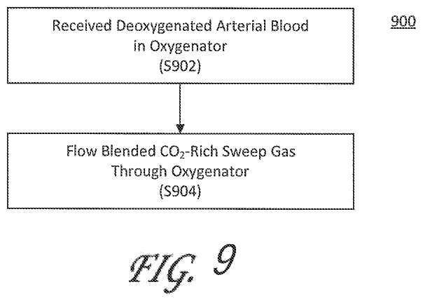

The plurality of fibers 554 may be arranged in a specific pattern to comprise the gas exchanger 550. Referring to FIG. 8, in some aspects, the gas exchanger 550 may be substantially cylindrical. Each of the fibers 554 may extend between the top and bottom opposing planar ends of the cylinder, such that all of the fibers 554 are disposed parallel to each other. In such an arrangement, the direction of flow of the sweep gas is preferably opposite the direction of flow of the blood. Referring to FIG. 8, for example, the sweep gas inlet may be disposed at one opposing planar end of the cylinder (e.g. the top end shown in the figure) with the sweep gas exhaust being disposed at the other opposing planar end of the cylinder (e.g. the bottom end shown in the figure), such that the sweep gas flows in direction D2 from the top to the bottom of the cylinder. The blood inlet port 504 can be arranged at the bottom end shown in the figure, and the blood outlet port 508 can be arranged at the top end shown in the figure, opposite the bottom end, such that the blood flows in a direction D1 from the bottom to the top of the depicted cylinder and opposite the flow of the sweep gas. This is advantageous because it allows for better and more efficient gas exchange between the blood and the gas-exchange fibers 554.

Referring to FIGS. 5-7, in another aspect of the disclosure, the fibers 554 may be arranged in a crisscross pattern or grid. Multiple fibers 554 may be disposed substantially parallel to each other in a planar arrangement. Multiple such arrangements may make up the gas exchanger 550, and the orientation of each planar arrangement may be the same as another planar arrangement, the same as all other planar arrangements, or different from other planar arrangements. Referring again to FIGS. 5-7, an exemplary portion of a gas exchanger 550 is shown having a first plane 570 that contains a plurality of fibers 554 and a second plane 572 adjacent to the first plane 570. The planes 570 and 572 are substantially the same, except that the fibers 554 of the second plane 572 are angled relative to the fibers 554 of the first plane 570.

While the second plane 572 is shown to be rotated 90 degrees or substantially perpendicular relative to the first plane, it will be appreciated that other relative angles between adjacent planes of fibers may be utilized. Any suitable number of planes 570, 572 may be arranged to form the gas exchanger 550.

The fibers 554 can be arranged such that a space 576 exists between adjacent fibers to allow the blood to flow through. The size of the space 576 may depend on one or more of the following parameters: the quantity of the fibers, the density of fibers, the flow rate of the blood, the flow rate of the sweep gas, the desired resistance within the oxygenator, and/or on other parameters that can affect gas exchange of the blood.

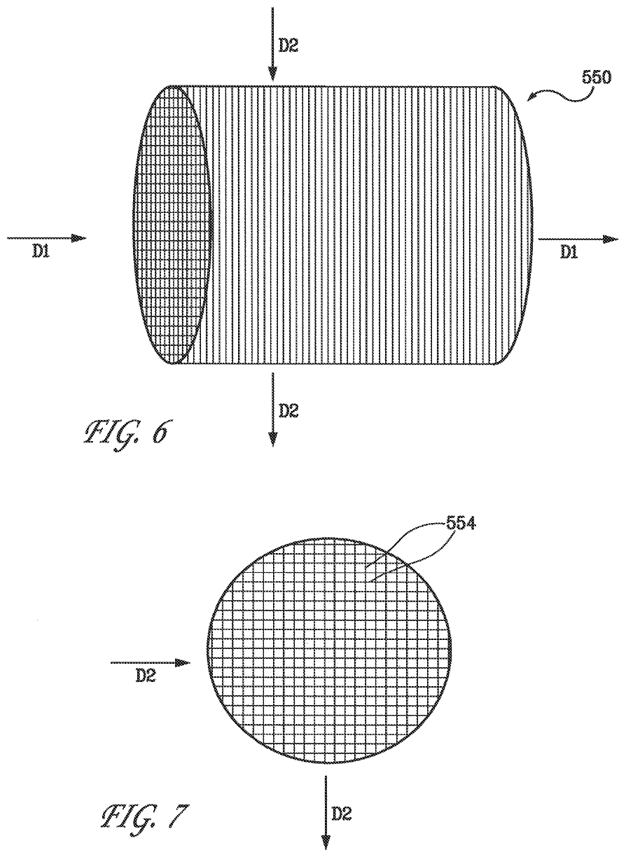

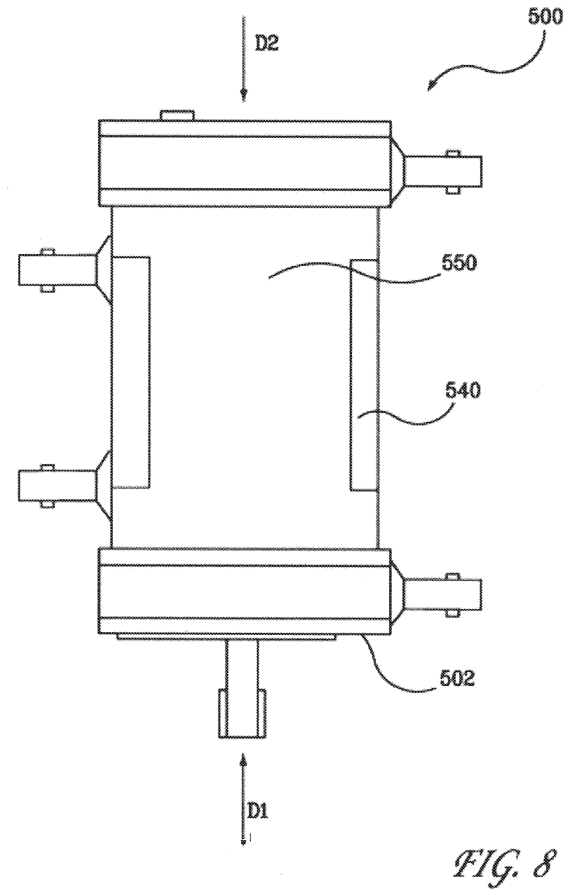

The gas exchanger 550 may include various shapes and configurations, such as cylindrical or cuboidal or parallelepiped. Referring to FIGS. 6-7, an arrangement of adjacent planes (e.g., plane 570 and plane 572 located at a 90-degree angle to plane 570) may be arranged as a cylinder having two opposing planar ends. Blood can enter the gas exchanger 550 at one of the planar ends, travel through the gas exchanger 550, and exit at the opposite planar end. Sweep gas can enter the gas exchanger 550 at the curved wall of the cylinder at one location and exit at another location on the curved wall.

In some aspects, referring again to FIG. 5 it may be advantageous to arrange the gas exchanger 550 to have a plurality of planes 570, 572 such that they form a cylinder as described above. Such a gas-exchanger would have a circular cross-section perpendicular to the blood flow direction D1. The circular cross-section eliminates corners, thus decreasing areas of higher turbulent flow and stagnant flows and helps maintain a more event (e.g., constant flow) throughout the gas exchanger 550. This reduces the likelihood of damage to the blood cells and decreases clot formation. Such an arrangement may be advantageous because it also decreases pressure within the oxygenator 500 and reduces resistance to flow. As noted above, due to the pumpless/passive nature of the system 10, it is important to have as low resistance to the blood flow as possible to allow the neonatal heart to pump blood through the oxygenation circuit 400 without stopping or significantly slowing the flow and without overexerting itself. If resistance of blood circuit is too low, blood flow can be non-invasively restricted on the tubing for the umbilical cord (440 in FIG. 1) prior to entering the oxygenator (between P1 and the oxygenator) if necessary to avoid starving neonatal blood circulation In some aspects, the gas exchanger 550, the housing 502, or any of the ports disclosed herein may be coated or lined with one or more anti-clotting, antithrombogenic, and/or non-thrombogenic materials, such as, but not limited to, immobilized polypeptide and heparin.

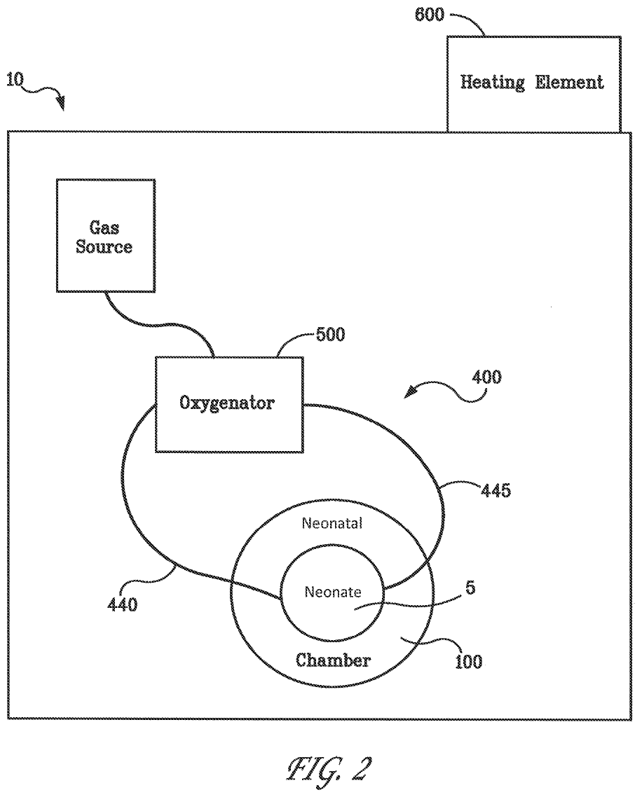

The system 10 may include a heating element 600 positioned therein and configured to heat the oxygenation circuit 400. The heating element 600 is not part of the oxygenator 500 itself. The heating element 600 may heat and maintain a desired temperature of the neonate 5, the environment in which the neonate 5 resides, the enclosure of the oxygenation circuit 400, and other components of the system 10. Referring to FIG. 2, an exemplary arrangement is depicted in which the heating element 600 is located separate from the oxygenator 500 and contacts the oxygenation circuit 400. FIG. 2 is an exemplary schematic showing an aspect of such an arrangement, and it will be appreciated that the heating element 600 may be disposed elsewhere and may be either directly adjacent or in indirect contact with the oxygenation circuit 400.

By maintaining the entire oxygenation circuit 400 within the desired temperature, there is no need to additionally heat the blood specifically as it flows to, through, and away from the oxygenator 500. The desired temperature can range from about 36 degrees Celsius to about 39 degrees Celsius. As such, a heating element 600 is neither needed nor desired within the oxygenator 500. Excluding the heating element 600 from the oxygenator 500 allows the oxygenator 500 to be smaller, require fewer fibers 554, impose less blood-flow resistance, and require a smaller amount of priming material to operate. It is important to note that an oxygenator within an extracorporeal circuit generally requires a heating element to maintain the desired temperature of the blood traveling therethrough. Failure to do this may result in damage to the blood, shock to the patient, or other health hazards. In the systems described throughout this application, the above drawbacks are eliminated by heating the entire system 10, or at least the oxygenation circuit 400, with the heating element 600. This allows exclusion of a heater from the oxygenator 500 itself, while maintaining the required temperature of the blood and sweep gas moving between the neonate 5 and the oxygenator 500.

As noted above, removing the otherwise-necessary heater from the oxygenator 500 allows for a smaller gas exchanger 550 and a smaller cavity 540, which in turn allows for a smaller necessary priming volume to operate the oxygenator 500. To start the oxygenation process, the oxygenator 500 must be filled with a suitable priming material. The larger the oxygenator 500, the greater the required minimum volume of priming material. In some aspects, when a neonate 5 is connected with the oxygenation circuit 400, the priming material comprises adult human blood (e.g., maternal blood or blood from a blood bank). Adult blood has different properties from neonatal blood, and it is preferred to minimize the impact of these differences. Priming the oxygenator 500 with adult blood results in hemodilution of the blood inside the neonate (i.e., the neonatal blood will mix with the adult blood used for priming). The greater the volume of the priming material, the greater the hemodilution. It may be advantageous to minimize the hemodilution within the neonate 5. By excluding a heater from the oxygenator 500 (in lieu of the heating element 600 within the system 10 or the oxygenation circuit 400), the total volume of the oxygenator 500 is decreased, thus requiring a smaller priming volume.

Further, decreasing the total size and volume of the oxygenator 500 also decreases the transit time of the blood as it moves through the oxygenator 500. Increased transit time may lead to thrombosis and clot formation, and decreasing the size of the oxygenator 500 decreases the transit time of the blood flowing therethrough, reducing the chance of clot formation. The blood flow rate through the oxygenator 500 may depend on the age and size of the neonate 5. For example, in some aspects, a neonate weighing approximately 500 grams would have a flow rate ranging from about 40 mL/min to about 60 mL/min. In some aspects, a 24-week-old neonate may have a flow rate ranging from about 60 mL/min to about 90 mL/min. The flow rate may be higher in a more developed and larger neonate and will depend, in part, on the weight of the neonate. Suitable flow rates may range from about 75 mL/kg/min to about 175 mL/kg/min.

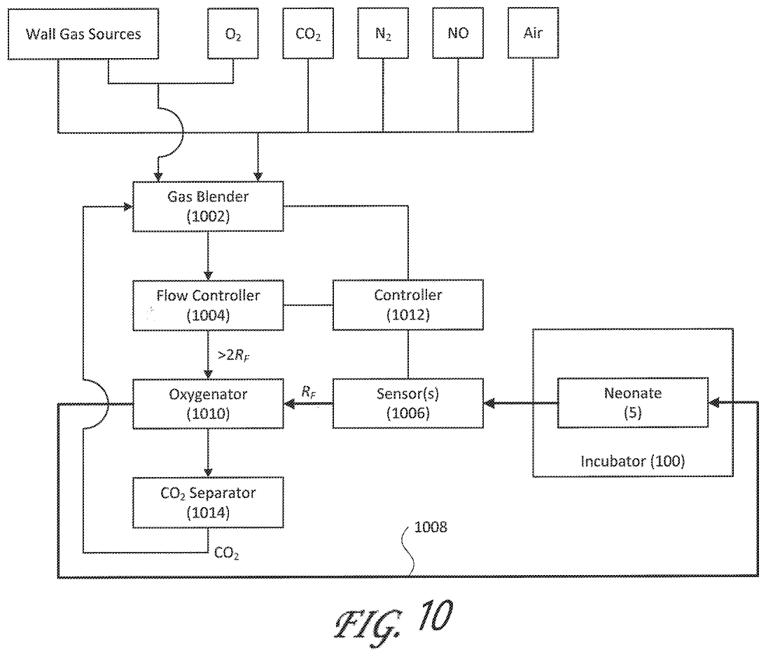

Referring now to FIGS. 9 and 10, another aspect of the invention provides systems and methods for controlling sweep gas flow and/or composition.

In one aspect such as that shown in FIGS. 9 and 10, there is a method for controlling the composition, gas flow rate, or combinations of a sweep gas comprising O₂, N₂, and CO₂ comprising the steps of receiving a deoxygenated arterial blood from a neonate in an oxygenator and flowing a blended CO₂ (higher than atmospheric pressure) rich sweep gas, which has an amount of CO₂ which is ranges from 0 to about 10 or from about 2 to about 5% by volume greater than the deoxygenated arterial blood wherein at least one sensor (shown in FIG. 10) is in electrical communication with a controller 1012 and flow controller 1004 to adjust the amount(s) of O₂, N₂, CO₂, and other components within the sweep gas via the gas blender 1002, optionally separating out the excess amount of CO₂ using the CO₂ separator, and providing the oxygenated blood to the neonate 5 in the neonatal chamber 100.

The sweep gas can be controlled to have an amount of CO₂ (e.g., by gas blender 1002) ranging from about 0 to about 10 or about 2 to about 5 and have a high flow rate (e.g., by flow controller 1004) relative to a neonatal blood flow rate R_{F} or the deoxygenated arterial blood. The sweep gas can further comprise CO₂, O₂, N₂, NO, and other gasses.

Neonatal blood flow rate R_{F} can be measured using a flow-rate sensor 1006 in communication with a blood-flow circuit 1008 from the neonate 5 within neonatal chamber 100 to the oxygenator and back to the neonate 5. The blood-flow circuit 1008 and the oxygenator 500 can be pumpless. That is, blood flow can be generated solely by the neonate's heart.

The neonatal blood flow can be measured in real-time or periodically. The neonatal blood flow can be measured using a flow sensor 1006 such as an ultrasonic flow sensor that can, e.g., be clamped-on a component of the blood-flow circuit such as tubing. Suitable sensors 1006 are available from Spectrum Medical of Gloucester, United Kingdom.

Applicant has discovered that preservation of normocapnia during extracorporeal support of a neonate 5 is particularly challenging. Without being bound by theory, it is believed that the low neonatal blood-flow rate (relative to adults) creates challenges in controlling sweep gas composition and flow (e.g., at very low flow rates). Embodiments of the invention overcome these challenges by “oversweeping”, i.e., using higher ratios of sweep gas to blood flow typically necessary to remove a desired quantity of CO2 so that the sweep gas flow rate can be better controlled (e.g., with greater precision and/or cheaper flow controllers).

In order to prevent the higher ratio of sweep gas from drawing too much carbon dioxide away from the neonatal blood (resulting in hypocapnia), the sweep gas contains an elevated proportion of carbon dioxide relative to that typically used in extracorporeal support, thereby maintaining normocapnia. Here, the composition of sweep gas can be adjusted by changing the CO2 level of the sweep gas to achieve the desired maintain normocapnia. The initial CO2 level of the sweep gas can initially be equal to a desired CO2 level in the neonatal blood. The CO2 level of the sweep gas can then be adjusted based on detected CO2 levels in the neonatal blood.

For example, some embodiments of a sweep gas contains from about 1% to about 6% CO₂ by volume based upon 100%, more preferably from about 3% to about 6% CO₂ by volume, e.g., from about 3.0% to about 3.5%, from about 3.5% to about 4.0%, from about 4.0% to about 4.5%, from about 4.5% to about 5.0%, from about 5.0% to about 5.5%, from about 5.5% to about 6.0%, and the like. Without being bound by theory, Applicant believes that such percentages of CO₂ in the sweep gas according to embodiments of the invention are two orders of magnitude greater than typically used in extracorporeal support, which typically blend pure oxygen with medical air. By way of comparison, atmospheric dry air contains 0.04% CO₂. In some embodiments, the sweep gas contains from about 0% to about 10%, about 10% to about 20%, about 20% to about 30%, about 30% to about 40%, or about 40% to about 50% O 2.

The particular sweep gas flow rate and CO2 composition can be adjusted based on the fiber surface area, transmissivity, and other properties of the oxygenator 500 and/or sensor readings (e.g., blood sensors 1006 and/or sweep-gas sensors as described and depicted in the context of FIGS. 1-8) to achieve and maintain desired blood oxygen and carbon dioxide levels.

For example, a controller 1012 can be communicatively coupled to each sensor (e.g., 1006) and gas blender 1002 and flow controller 1004. The principles of how to use feedback (e.g., from blood oxygen and carbon dioxide sensors) in order to modulate operation of a component are described, for example, in Karl Johan Astrom & Richard M. Murray, Feedback Systems: An Introduction for Scientists & Engineers (2008). For example, the concentration of CO₂ in the sweep gas entering the oxygenator 500 may change over time (e.g., by feedback or by a non-feedback-based model) to reflect “wear” of an oxygenator, growth of the neonate, and the like.

Without being bound by theory, Applicant believes that oversweeping with a high flow rate of CO₂-rich sweep gas advantageously avoids the typical need to periodically “sigh” an oxygenator during ECMO by increasing the gas flow rate to remove condensation from the oxygenator. Although sighing is tolerated in adult ECMO, the relatively small proportions of a neonate are less able to tolerate such an increase in gas flow without causing hypocapnia.

Still referring to FIG. 10, another aspect of the invention provides systems and methods for capturing CO₂ for use in producing CO2-rich sweep gas. In one embodiment, a CO₂separator 1014 (e.g., a CO₂-selective membrane such as a glassy polymeric membrane, metal-organic framework (MOF), zeolitic-imidazolate framework (ZIF), and the like, a cryogenic distillation device and the like) is employed between the oxygenator exhaust 516 and the CO₂input of the gas blender 1002 and the separated CO₂ is recycled as an input to the gas blender 1002.

Although preferred embodiments of the invention have been described using specific terms, such description is for illustrative purposes only, and it is to be understood that changes and variations may be made without departing from the spirit or scope of the following claims.

The entire contents of all patents, published patent applications, and other references cited herein are hereby expressly incorporated herein in their entireties by reference.

In particular, U.S. Pat. Nos. 10,864,131 and 10,751,238 and U.S. Patent Application Publication Nos. 2019/0380900 further describe extracorporeal systems and International Publication No. WO 2020/210275 further describes oxygenators, each suitable for combination with the systems and method described herein.

1. A method of oxygenating neonatal blood, the method comprising:
(a) receiving deoxygenated arterial blood from an umbilical cord of a neonate within a pumpless extracorporeal circuit comprising a membrane oxygenator, wherein the deoxygenated arterial blood has a blood-flow rate R_{F}; and
(b) flowing a blended sweep gas through the membrane oxygenator at a blended sweep gas flow rate of at least 2 R_{F} or greater,
wherein the blended sweep gas comprises oxygen and at least about 1% carbon dioxide by volume.

2. The method of claim 1, wherein the blended sweep gas further comprises one or more selected from the group consisting of: ambient air at sea level, nitrogen, and nitric oxide.

3. The method of claim 1, wherein the blended sweep gas comprises a sufficient partial pressure of carbon dioxide to maintain normocapnia in the neonate's venous blood.

4. The method of claim 1, wherein the blended sweep gas flow rate is between about 2 R_{F} and about 20 R_{F}.

5. The method of claim 1, wherein the blended sweep gas flow rate is between about 2 R_{F} and about 16 R_{F}.

6. The method of claim 1, further comprising:
(a′) measuring the blood-flow rate R_{F}.

7. The method of claim 1, wherein the blended sweep gas flow rate is between about 120 mL/minute and about 800 mL/minute.

8. The method of claim 1, wherein the blended sweep gas comprises between about 3% and about 6% carbon dioxide by volume.

9. The method of claim 1, wherein at least a portion of the carbon dioxide of the blended sweep gas was previously recovered from exhaust gas from the membrane oxygenator.

10. The method of claim 1, further comprising receiving with a gas blender, a first gas from a first source and a second gas from a second source, the first gas different from the second gas, and blending the first gas and the second gas to form the blended sweep gas.

11. The method of claim 10, wherein the first source is a first gas tank and the second source is a second gas tank.

12. The method of claim 10, wherein the first source is a wall line and the second source is at least one of a second wall line and a gas tank.

13. A pumpless oxygenation circuit comprising:
a mixer adapted configured to:
receive oxygen-containing gas from an oxygen source;
receive carbon-dioxide-containing gas from a carbon-dioxide source;
output a blended sweep gas comprising oxygen and at least about 1% carbon dioxide by volume;
a flow controller fluidically coupled to the output of the mixer, the flow controller adapted to permit a blended sweep gas flow rate of the blended sweep gas through the flow controller;
a membrane oxygenator adapted for connecting to the flow controller and a pumpless neonatal blood circuit; and
a blood-flow sensor positioned along the pumpless neonatal blood circuit, the blood-flow sensor adapted and configured to measure a neonatal-blood-flow rate R_{F},
wherein the flow controller controls the blended sweep gas flow rate to be at least 2 R_{F} or greater

14. The pumpless oxygenation circuit of claim 13, further comprising:
a controller communicatively coupled with the flow controller and the blood-flow sensor.

15. The pumpless oxygenation circuit of claim 14, further comprising:
a blood-gas sensor positioned along the pumpless neonatal blood circuit, the blood-gas sensor communicatively coupled to the controller;
wherein the controller controls the mixer to titrate a composition of the blended sweep gas to reflect neonatal blood-gas values and provide a sufficient partial pressure of carbon dioxide to maintain normocapnia.

16. The pumpless oxygenation circuit of claim of claim 13, wherein the blended sweep gas flow rate is between about 2 R_{F} and about 20 R_{F}.

17. The pumpless oxygenation circuit of claim of claim 13, wherein the blended sweep gas flow rate is between about 2 R_{F} and about 16 R_{F}.

18. The pumpless oxygenation circuit of claim of claim 13, wherein the blended sweep gas flow rate is between about 120 mL/minute and about 800 mL/minute.

19. The pumpless oxygenation circuit of claim of claim 13, wherein
the blended sweep gas comprises between about 3% and about 6% carbon dioxide by volume.

20. A neonatal extracorporeal support system comprising:
a neonatal chamber adapted and configured hold a neonate in a physiological saline solution;
a neonatal blood system adapted and configured for coupling to a plurality of blood vessels of a neonate; and
the pumpless oxygenation circuit according to claim 13.
